(19) United States
(12) Patent Application Publication
(10) Pub. No.: US 2012/0136247 A1
(43) Pub. Date: May 31, 2012

(54) Methods of Transvascular Retrograde Access Placement and Devices for Facilitating the Placement

(76) Inventor: Lakshmikumar Pillai, Morgantown, WV (US)

(21) Appl. No.: 12/366,517

(22) Filed: Feb. 5, 2009

Related U.S. Application Data
(63) Continuation-in-part of application No. 11/424,131, filed on Jun. 14, 2006.
Application No. 11/424,131 is a continuation of application No. 11/381,229, filed on May 2, 2006, now abandoned.

(51) Int. Cl. A61M 25/098; A61B 6/00; A61M 25/09; A61M 25/06

(52) U.S. Cl. 600/433; 604/22; 604/528; 604/529

(57) ABSTRACT

The invention relates to methods of transvascular retrograde access placement into a central blood vessel and to devices that facilitate these methods. The method includes positioning a vascular catheter within the vessel such that its distal end faces a desired exit site on the vessel wall, passing a penetrating device from the catheter through the vascular exit site and skin of the patient; and passing a secondary vascular catheter through the exit site and into the central blood vessel. In another aspect, the method includes penetrating outward from within the vessel to form a pass-through site on a wall of the vessel that exits the patient; and placing the vascular access device into the vessel through the pass-through site. Typical vessels that may be accessed include central veins, but may include other veins as well as arteries. Devices that facilitate the method include a stiff intravascular guidewire with a distally-mounted penetrating device such as a needle, and a two-channel vascular catheter with an angled tip.

### CROSS REFERENCE TO RELATED APPLICATIONS

This application is a continuation-in-part of U.S. application Ser. No. 11/424,131 of Pillai, entitled “Methods of Transvascular Retrograde Access Placement and Devices for Facilitating Therein”, filed Jun. 14, 2006; which application is a continuation of U.S. application Ser. No. 11/381,229, filed May 2, 2006. Both applications are incorporated by reference as if fully set forth herein.

### FIELD OF THE INVENTION

The present invention relates to methods and devices directed toward providing transvascular retrograde access placement in central vessels. More particularly, these methods and devices direct an initial passage of a guidewire from the inside of the vessel to the outside, followed by guided insertion of a catheter over the guidewire into the vessel.

### INCORPORATION BY REFERENCE

All publications, patents and patent applications mentioned in this specification, either by an inventor common to this application or by other inventors, are herein incorporated by reference to the same extent as if each individual publication, patent, or patent application was specifically and individually indicated to be incorporated by reference.

### BACKGROUND OF THE INVENTION

Gaining direct access by way of a catheter to a central vein (one which goes directly to the heart) is a common procedure that is useful for a number of medical needs, including providing fluids and nutrition, administering drugs, and allowing access to the heart for cardiovascular measurements or the implantation of devices such as pacemakers. Conventional approaches for performing central venous catheterization, as shown in FIG. 1, generally involve the placing of catheters, needles and/or wires through a percutaneous entry site 1 into a jugular vein 14 or a subclavian vein 16 and subsequent central venous cannulation. This method involves inherent risks with potentially serious consequences and adverse effects to the patient due to the technique's essentially blind puncture through the skin and percutaneous tissue (i.e., from the outside of the skin to the inside of the central vein) overlaying an accessible site of the vein. More specifically, conventional techniques, with or without fluoroscopic guidance, involve percutaneous puncture of either the jugular or subclavian vein with a hollow needle, and the passage of a guidewire into the punctured vein through the needle. Thereafter, the guidewire assists with the insertion of a vascular catheter, which then ultimately replaces the guidewire. Theses central veins are deep structures, and cannot be visualized without imaging technology. The percutaneous puncture site generally is determined by anatomic landmarks (“dead reckoning”) or, less commonly, with the aid of transcutaneous ultrasound.

While this conventional technique is usually accomplished with few or any complications and minimal pain to the patient, the technique, due to the blind percutaneous puncture, inherently carries significant risks. These risks include potentially disabling or life-threatening injuries such as injury to adjacent vascular and lymphatic structures or nerves, occurrence of stroke secondary to vascular injury, or occurrence of pneumothorax or hemothorax. The risk of eventualities such as these are more likely when the technique is performed on children or on adult patients with challenging anatomy or conditions, such emaciation or morbid obesity.

Safer and more cost-efficient alternative approaches to central vein access that obviate the need for blind percutaneous vein puncture would be a welcome addition to the possible approaches available to patients requiring central vein access.

### SUMMARY OF THE INVENTION

The present invention provides methods for performing transvascular retrograde access placement in a central blood vessel as well as devices that facilitate the method. Embodiments of the method include positioning a vascular catheter within the central blood vessel such that a portion of the vascular catheter faces a desired exit site on a wall of the central blood vessel, passing a penetrating device from the vascular catheter through the desired exit site on the wall of the central blood vessel and skin of the patient, and passing an end of a secondary vascular catheter through the exit site and into the central blood vessel. Some embodiments of the method, prior to the positioning step, further include inserting the vascular catheter into a primary blood vessel of the patient and advancing the vascular catheter to the desired exit site on the wall of the central blood vessel. Exemplary central blood vessels include a jugular vein or a subclavian vein; exemplary primary blood vessels, which provide an approach to the central vessel, include a femoral vein or an antecubital vein.

Some embodiments of the method, prior to the passing the penetrating device through the central vein exit site, further include advancing the penetrating device through the vascular catheter to the desired exit site. In some of these embodiments, the catheter includes more than one channel, and advancing the penetrating device through the vascular catheter includes advancing through a one of the channels.

Some embodiments of the method, prior to passing the penetrating device through the exit site, further include forming an opening at the desired exit site in the wall of the central blood vessel with the penetrating device. Exemplary penetrating devices may include any of a needle, a radiofrequency knife, a laser, a high frequency ultrasound device, or an electrosurgical device. Some embodiments of the invention include supporting the penetrating device on an end of a stiff intravascular guidewire.

In some embodiments of the method, passing an end of the secondary vascular catheter through the exit site of the central vein includes passing the secondary vascular catheter over the penetrating device. In other embodiments of the method, the penetrating device is supported on an end of a guidewire but then removed after the penetrating device is passed through the exit site and skin, such that passing an end of the secondary vascular catheter through the exit site of the central vein includes passing it over the guidewire.

In some embodiments of the method, after passing the penetrating device through the exit site, the method further includes pulling the vascular catheter and the penetrating device through the skin; and removing the vascular catheter from the patient while leaving the guidewire in position. In some of these embodiments, the method further includes removing the vascular catheter from the patient.

Some embodiments of the method include providing a diagnostically-opaque substance into a vascular system prior to the positioning step. Still other embodiments include visualizing diagnostically-opaque markers on the vascular catheter prior to passing the penetrating device through the desired exit site.

In some embodiments, the vascular catheter used in the method comprises an angled-tip. The vascular catheter used in the method may further include a first channel and a second channel, and in these embodiments, the method may further include introducing an angled-tip stiff obturator into the vascular catheter through the first channel. In some of these embodiments, a distal tip of the angled-tip stiff obturator has a preferred angle with respect to a proximal length of the obturator, and advancing the obturator through the vascular catheter includes advancing the obturator to a distal end of the catheter; and altering an angle of the distal end of the catheter toward the preferred angle of the angled tip.

Embodiments of the invention also include methods of performing vascular access placement through an exit-formed opening in a blood vessel of a patient. This method embodiment includes positioning a vascular catheter within the blood vessel such that a portion of the vascular catheter faces a desired exit site on a wall of the blood vessel, passing a penetrating device from the vascular catheter through the desired exit site on the wall of the blood vessel and a skin site of the patient over the desired exit site, and passing an end of a secondary vascular catheter through the exit site and into the blood vessel. In various embodiments of this method, the blood vessel may be a vein, a central vein, or an artery.

In another aspect, embodiments of the invention include a method of placing a vascular access device in a central blood vessel of a patient that includes penetrating outward from within the vessel to form a pass-through site on a wall of the vessel that exits the patient; and placing the vascular access device into the vessel through the pass-through site.

In some embodiments of this aspect of the method, following the penetrating step, the method includes withdrawing the penetrating device back through the pass-through site into a vascular catheter; and advancing a guidewire from the catheter through the pass-through site to emerge through skin overlaying the blood vessel. In these embodiments of the method, the placing step may include passing the vascular access device over the guidewire. In other embodiments of this aspect of the method, the penetrating step includes advancing a stiff intravascular guidewire with a distally-mounted penetrating device through a distal tip of a vascular catheter. In these embodiments, the placing step includes passing the vascular access device over the guidewire.

Further, embodiments of this aspect of the method may include, positioning a distal tip of a vascular catheter within the vessel to face a desired pass-through site on a wall of the vessel. In these embodiments, the positioning step may include advancing the vascular catheter to the pass-through site from a direction (such as from the direction of the heart), and wherein the placing step comprises placing the vascular access device into the vessel oriented in that direction (such as toward the heart).

Embodiments of the invention include various devices adapted particularly for use in the method as summarized above. A device suitable for use in performing transvascular retrograde access placement includes a stiff intravascular guidewire and a vascular wall penetrating device supported on a distal end of the guidewire. In some embodiments, the vascular wall penetrating device is removably coupled to the guidewire, as for example, with a threaded mechanism.

Various types of tissue-penetrating devices may be used by embodiments of the stiff, penetrating guidewire, as for example, a needle, a radiofrequency knife, a laser, a high frequency ultrasound device, or an electrosurgical device. Some embodiments of a needle as a penetrating device range in length from at least about 1 cm to about 2.5 cm, such length generally being sufficient to penetrate outwardly from the jugular vein, through intervening tissue, and exit from the skin. Embodiments of the invention that are applied to vascular sites other than the jugular vein may vary from these dimensions as may be appropriate for the anatomy surrounding the vascular site. Embodiments of the stiff penetrating guidewire have a length that varies between about 150 cm and about 300 cm; particular embodiments have a length that varies between about 225 cm and about 275 cm. In some embodiments, the guidewire has a lubricious coating.

Embodiments of the invention also include a device suitable for use in performing transvascular retrograde access placement comprising a vascular catheter comprising an angled tip and two channels. In some of these embodiments, at least one of the two channels has an inner diameter of at least about that of an external diameter of a 22-gauge needle. In some embodiments, the angled-tip includes an angled portion with an angle of about 30 to about 60 degrees with respect to the longitudinal axis of the catheter. In some of these embodiments, the angled portion is sufficiently elastic to become substantially linear when the catheter is within the confines of a blood vessel and to elastically assume a more acute angle when a curved obturator with an angled portion is advanced into the angled portion of the vascular catheter

Some embodiments of the angled-tip vascular catheter have one or more diagnostically-opaque markers attached to or incorporated into the catheter structure, particularly the distal portion of the catheter, to allow their visualization by diagnostic imaging methods. In some embodiments, these markers are positioned within or about the angled-tip of the angled-tip vascular catheter.

### BRIEF DESCRIPTION OF THE FIGURES

FIG. 1 shows a conventional technique of the prior art of performing upper trunk central venous catheterization.

FIG. 2 shows a vascular sheath inserted into a femoral vein of the central venous system of a patient.

FIG. 3 shows a general use guidewire passed through a femoral venous vascular sheath and the venae cavae to a jugular vein.

FIG. 4 shows an angled-tip vascular catheter passed over a general use guidewire and to a jugular vein.

FIG. 5 shows an embodiment of a needle-tipped guidewire of the present invention inserted, via the femoral venous vascular sheath, through the angled-tip vascular catheter and used to puncture the wall of the jugular vein.

FIG. 6 shows a secondary vascular catheter passed over the needle-tipped guidewire outside the mid-neck skin surface and an end of the secondary vascular catheter inserted into the jugular vein.

FIG. 7 shows an end of the secondary vascular catheter placed in the jugular vein after the needle-tipped guidewire and femoral vascular sheath have been removed from the venous system of the patient.

FIG. 8 shows an embodiment of the method wherein an angled catheter enters the vascular system through an antecubital vein and is advanced to a desired exit site in the jugular vein, where a tissue-penetrating device is delivered through the catheter to form a pass-though into which a retrograde jugular vein access device may be placed.

FIGS. 9A-9D show various embodiments of a stiff intravascular guidewire with a penetrating device mounted at the distal end. FIG. 9A shows a needle-tipped guidewire.

FIG. 9B shows a guidewire with a distally-mounted radiofrequency knife.

FIG. 9C shows a guidewire with a distally-mounted electrosurgical device.

FIG. 9D shows a guidewire with a distally-mounted high frequency ultrasound emitter.

FIG. 9E shows a guidewire with a distally-mounted laser device.

FIG. 10A shows an embodiment of an angled-tip vascular catheter of the present invention with a main channel and a second channel, and a curved-tip stiff obturator being passed through a main channel.

FIG. 10B shows an embodiment of an angled-tip vascular catheter with a second channel having a curved-tip stiff obturator passed through the main channel of the vascular catheter and a needle-tipped guidewire of the present invention being passed through the second channel.

FIG. 11A shows an embodiment of an angled-tip vascular catheter of the present invention that includes diagnostically-opaque markers positioned about the angled-tip.

FIG. 11B shows an angled-tip and diagnostically-opaque markers positioned about the angled-tip of an embodiment of a vascular catheter of the present invention.

FIG. 12 shows the adjustability of the degree of angle of an angled-tip of a vascular catheter of the present invention adjusted by advancing or withdrawing the curved-tip stiff obturator.

FIGS. 13A-13L show an embodiment of a method that makes use of a two-channel catheter, with a focus on the activity that occurs at the site of exit from the jugular vein and re-entry there into. FIG. 13A shows the approach of a general use guidewire to the vicinity of a desired exit site in a jugular vein.

FIG. 13B shows the approach of an inventive angled-tip two-channel vascular catheter by way of its primary channel over a general use guidewire to the vicinity of the desired exit site in the jugular vein. The tip of the vascular catheter has an angle of about 45 degrees from the central longitudinal axis of the catheter, and may be diagnostically visualizable by the radiopaque marking.

FIG. 13C shows the two-channel angled-tip vascular catheter with the general use guidewire having been withdrawn and a solid wall obturator with a curved tip having been advanced forward in the same primary channel toward the distal end of the vascular catheter.

FIG. 13D shows a solid obturator having advanced to the distal end of the primary channel of the two-channel vascular catheter, the curved tip of the catheter now substantially conforming to the approximate 90 degree angle of the obturator, and perpendicularly facing the wall of the jugular vein at the desired exit site.

FIG. 13E shows a stiff guidewire with a penetrating device on the distal end having been advanced to the distal end of the secondary channel of the two-channel vascular catheter.

FIG. 13F shows the penetrating device mounted on the distal end of the stiff guidewire advancing out of the second channel of the two-channel vascular catheter, and penetrating outwardly through the wall of the jugular vein into neck muscle and subcutaneous tissue.

FIG. 13G shows the penetrating device penetrating further outward through the skin of the neck and from the body, the stiff intravascular guidewire trailing behind.

FIG. 13H shows the penetrating device and guidewire positioned outside the body, the two-channel vascular catheter now having been withdrawn back through the jugular vein and eventually to exit through its femoral vein site of entry.

FIG. 13I shows a secondary vascular catheter being passed over the stiff intravascular guidewire, now outside the body, in a retrograde direction, from the distal end of the guidewire toward the proximal end. In some embodiments of the method, the penetrating device is removed from the distal end of the guidewire at this point.

FIG. 13J shows the secondary vascular catheter entering the jugular vein in a retrograde direction, in the direction of venous blood flow, over the stiff intravascular guidewire.

FIG. 13K shows the secondary vascular catheter in place in the jugular vein and positioned to act as a vascular access port, the stiff intravascular guidewire having been withdrawn from the jugular vein, ultimately to exit through its femoral vein site of entry.

FIG. 13L shows the secondary vascular catheter in place in the jugular vein as a vascular access port, and as an example of use, a syringe centrally administering drug through the central vein access port.

FIGS. 14A-14F show an embodiment of a method that makes use of a single-channel catheter, with a focus on the activity that occurs at the site of exit from the jugular vein and re-entry there into. FIG. 14A shows the approach of a general use guidewire to the vicinity of a desired exit site in a jugular vein.

FIG. 14B shows the approach of a vascular catheter over a general use guidewire to the vicinity of the desired exit site in the jugular vein. The tip of the vascular catheter has an angle of about 45 degrees from the central longitudinal axis of the catheter, and may be diagnostically visualizable by the radiopaque marking.

FIG. 14C shows the angled-tip vascular catheter with the general use guidewire having been withdrawn and a stiff guidewire with a penetrating device on its distal end being advanced to the distal end the curved tip vascular catheter.

FIG. 14D shows the angled vascular tip catheter rotating toward a desired exit site on the wall of the jugular vein, the site of exit on an exterior facing aspect of the vein, the rotation being driven by torque applied at the proximal end of the catheter.

FIG. 14E shows the penetrating device mounted on the distal end of the stiff guidewire advancing out of the vascular catheter, and penetrating outwardly through the wall of the jugular vein into neck muscle and subcutaneous tissue.

FIG. 14F shows the penetrating device penetrating further outward through the skin of the neck and from the body, the stiff intravascular guidewire trailing behind. (Hereinafter, the method proceeds as depicted in FIGS. 13H-13I.)

### DETAILED DESCRIPTION OF THE INVENTION

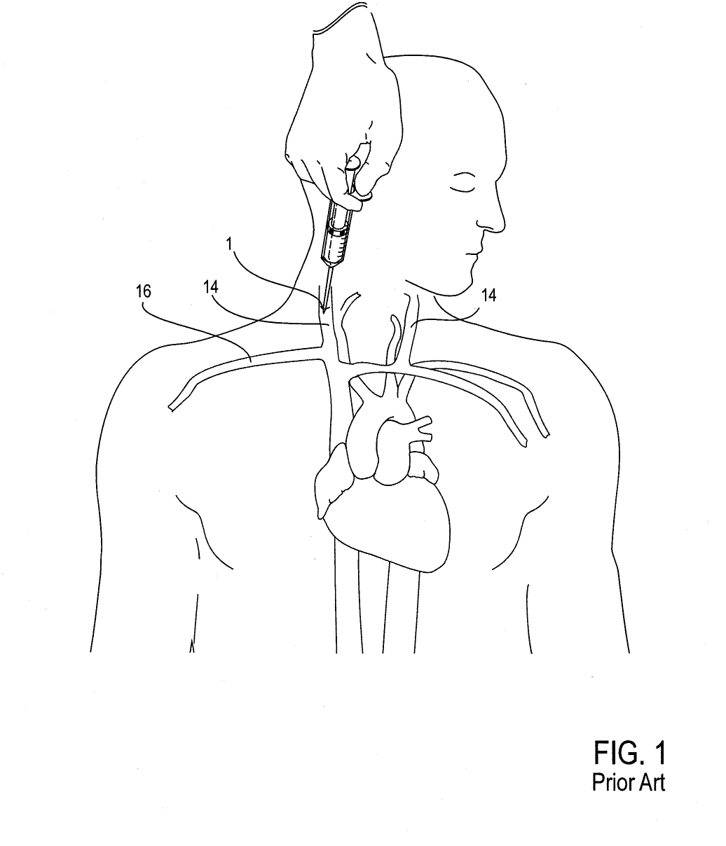

Embodiments of a method of the transvascular retrograde access placement, as provided herein, include the puncturing of a central blood vessel from the inside of the vessel with a penetrating device, such as a needle or other similarly configured device, and exiting that penetrating device from a patient through the skin. In some embodiments of the method, that penetrating device is supported at the distal end of a stiff intravascular guidewire; in other embodiments, the method may make use of an elongate needle which is then withdrawn prior to the passing a guidewire through the opening formed by the needle. By either approach, i.e., using an elongate needle or using a penetrating device at the distal end of a guidewire, the method involves passing outward through the skin, such passing originating from within the blood vessel. By such an inside-to-outside approach, the ability of a surgeon to precisely determine the location of a pass-through site in the vascular wall is substantially enhanced over prior art methods that rely on a conventional outside-to-inside approach, as shown in FIG. 1.

After the penetrating device penetrates through the skin to the exterior of the body, a vascular catheter may be inserted into the central blood vessel by means of the passing the catheter over the penetrating device in a retrograde direction. In embodiments where the penetrating device is supported by a stiff intravascular guidewire, the vascular catheter passes over the guidewire, either after passing over the penetrating device or instead of passing over the penetrating device in the event that the penetrating device is removed after effecting passage through the skin and being pulled free therefrom. Exemplary penetrating devices or technologies other than a needle may include, for example any of a radiofrequency knife, laser, high frequency ultrasound device, or an electrosurgical device. Although exemplary penetrating devices as described and as depicted herein, are typically represented as a needle or a needle-tipped guidewire, it should be understood that embodiments of the invention include the use of any of these tissue penetrating elements.

Embodiments of the method and devices for implementing the method are directed toward various regions of the vascular system, in accordance with particular medical indications. One particular use of the methods of the invention is for central vein access, in which a central vein, such as a jugular vein or subclavian vein is accessed. The initial approach to the central vein, by way of a guidewire, followed by a vascular catheter, is by way entry into a primary vein, such as femoral vein or antecubital vein. Thereafter, a penetrating element, positioned by advancement from the primary vein site of entry to a desired site of exit in the central vein, creates an opening through the vessel wall and overlaying skin. That site of exit, in turn, becomes the site of re-entry for a central vein catheter. Inasmuch as what is initially formed as an exit site from the vessel can later be used as a site for entry for a central vein catheter, the site of opening/entry may also be neutrally referred to as a vascular pass-through site.

Embodiments of the invention, while generally described and depicted herein in the context of providing retrograde access into a central vein through an opening originally formed as an exit from the central vein, the invention may also be generally understood as providing methods of vascular entry through an exit-formed opening in blood vessels other than a central vein. This method embodiment includes positioning a vascular catheter within a blood vessel such that a portion of the vascular catheter faces a desired exit site on a wall of the blood vessel, passing a penetrating device that is advanced from the vascular catheter through the desired exit site on the wall of the blood vessel and a skin site of the patient overlaying the desired exit site, and passing an end of a secondary vascular catheter through the exit site and into the blood vessel. In various embodiments of this method, the blood vessel may be a vein, a central vein, or an artery. An example of an artery that is particularly difficult to access externally is the subclavian artery, thus there may be advantages to accessing the subclavian artery by such methods as described herein.

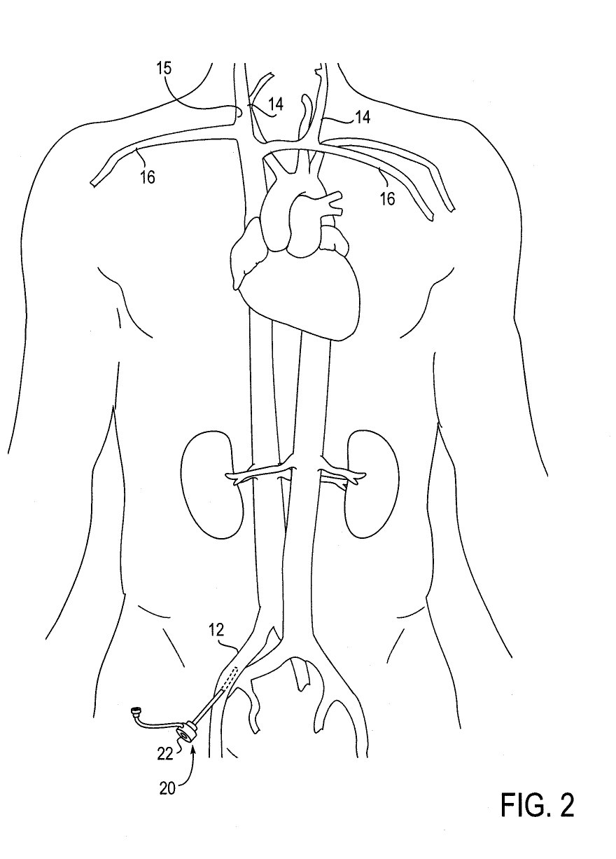

Some embodiments of the methods of transvascular retrograde access placement of the present invention are performed in a jugular 14 or subclavian 16 vein, although it is contemplated by the present invention that these methods may be performed through the puncture or penetration of any central blood vessel, the initial penetration occurring from the inside of the vessel, toward the outside. As shown in FIGS. 2-14 and as described below, various embodiments of devices of the present invention include a stiff, tissue-penetrating guidewire 50 having or supporting a needle-tip portion 52 as an exemplary penetrating device; and an angled-tip vascular catheter 40. Embodiments of the vascular catheter may include two channels (a two-channel catheter 41), for example a primary channel 45 and a second channel 46, and the catheter may further include one or more diagnostically-opaque markers 44 placed within or about the angled-tip 42. Diagnostically-opaque markers typically refer to markers 44 that can be diagnostically recognized through advanced diagnostic imaging technologies, as for example, high-resolution ultrasound, X-ray, CAT-scan, fluoroscopy, and/or magnetic resonance imaging (MRI), and which provide visual information as to the position of the tip of the vascular catheter in the blood vessel. Further, in some areas, the imaging technology has been coupled with navigational capability, such as magnetic navigation as enabled by MRI. The use of any of such diagnostic or navigational capability may be used in conjunction with fundamental aspects of the present invention, and are included within the scope of the invention. In some embodiments, diagnostically opaque markers may also refer to a substance introduced into the vascular system to enable visualization of the tip of the vascular catheter.

Embodiments of the method of transvascular retrograde access placement of the present invention generally include performing cannulation of a primary blood vessel, as, for example, a femoral vein 12 or antecubital 18 vein; inserting an angled-tip vascular catheter 40, as may be modified by the invention as described further below, into the blood vessel; and positioning the angled-tip vascular catheter 40 inside a central blood vessel, as, for example, a jugular 14 or subclavian 16 vein, of the vascular system. The method continues with the passing of a needle or other similarly functional penetrating device, or a guidewire 50 modified so as to include a needle-tip portion 52, through or about the vascular catheter 40; and penetrating through the wall 15 of that central blood vessel with the needle, passing the needle through the subcutaneous tissue, and exiting the needle through the patient's skin. The vascular catheter 40 may then be removed from the patient, leaving the guidewire in place. A secondary vascular catheter 60 may then be passed over the needle from the outside of the skin such that a distal end of the secondary vascular catheter 60 is placed within the punctured central blood vessel while a proximal end of the secondary vascular catheter 60 remains exposed from the skin of the patient. The needle is then fully removed from the patient at either the exit site from the central blood vessel or from the initial cannulation site in the primary blood vessel.

According to the herein described methods of the present invention for performing transvascular retrograde access placement, these methods may further include using advanced diagnostic imaging technologies such as fluoroscopy, by way of example. By providing a diagnostically-opaque substance, usually an iodinated vascular contrast material, to the vascular system of the patient so that the vascular system is visible through advanced diagnostic imaging technologies, the level of safety is enhanced in performing these methods. This diagnostically-opaque substance generally is introduced into the vascular system prior to the insertion of the vascular catheter 40 into the primary blood vessel and, again, in the vicinity of a central blood vessel immediately prior to the puncture of a wall 15 of the central blood vessel.

The primary blood vessel cannulation may be achieved by means of the Modified Seldinger Technique, wherein the desired vessel or cavity is punctured with a sharp hollow needle; a round-tipped guidewire (the first of several guidewires used in this procedure) is then advanced through the lumen of the needle, and the needle is withdrawn. An introducer is then inserted over the round-tipped guidewire, and into the vessel; a “sheath” or blunt cannula is passed through the introducer; and the guidewire and introducer are then withdrawn. The sheath can then be used to introduce catheters into the vessel.

In accordance with the methods of the present invention, the Modified Seldinger Technique typically is performed with an 18-gauge hollow needle about 2⅞ inches in length, and a 5 cc syringe secured to the end of the 18-gauge needle opposite of the needle-tip. Typically, a femoral vein 12 is percutaneously cannulated with the needle. Once blood from the femoral vein 12 is aspirated into the syringe, the syringe is removed while the needle is held in place. Through this needle, a short guidewire, such as a J-tip wire measuring about 0.035 inches in diameter and about 20 centimeters in length may be advanced up the femoral vein 12 and into the iliac vein. The needle is then removed while the short guidewire is held in place. Thereafter, as shown in FIG. 2, a vascular sheath 20, generally measuring about 10 centimeters in length and typically but not necessarily including a removable stiff introducer and a hemostatic valve 22, is advanced over the short guidewire and into the femoral vein 12. The vascular sheath 20 is typically a 5 French (F) sheath, but may be a 6F or a 4F sheath, or another similarly sized and configured sheath. (French/3.14=inner diameter of a sheath or catheter in millimeters).

Then, with the vascular sheath 20 being substantially introduced into the patient, the short guidewire and the stiff introducer are removed and the vascular sheath 20 may be flushed by inserting heparinized saline solution into the vascular sheath 20 through the hemostatic valve 22. In various embodiments of the methods of the present invention, the Modified Seldinger Technique may be performed with other similarly sized and configured needles, syringes, sheaths, and/or wires.

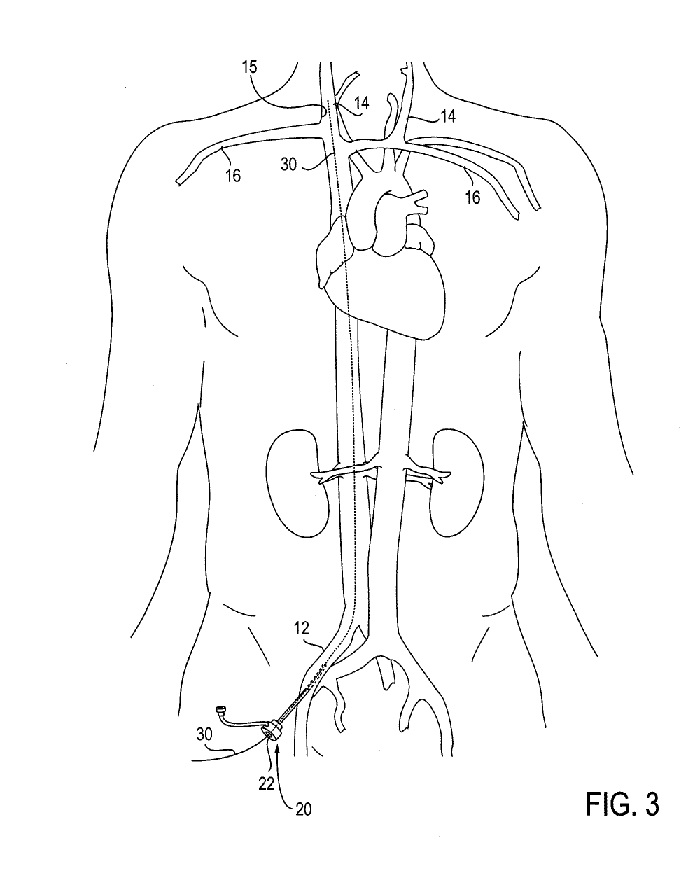

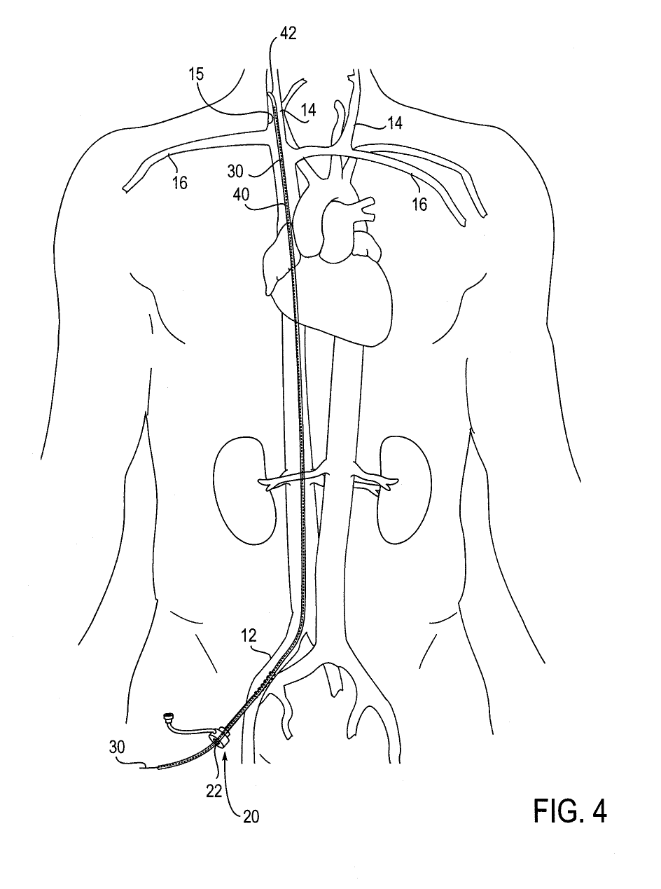

Thereafter in furtherance of embodiments of the method, as shown in FIG. 3, a general use guidewire 30 typically measuring about 0.035 inches in diameter may be passed through the vascular sheath 20 and ultimately positioned in a jugular vein 14 in the vicinity of a desired exit and reentry site (or pass-through site) 25 for central vein access. Following the insertion and positioning of the general use guidewire 30, an inventive angled-tip vascular catheter 40 may be passed over the general use guidewire 30, as shown in FIG. 4, after which the general use guidewire 30 is removed.

Embodiments of the inventive angled-tip vascular catheter 40 are typically a 5F catheter, but may be a 4F catheter, or another similarly sized and configured catheter. Embodiments of a vascular catheter 40 generally measure about 100 centimeters in length and include or support an angled-tip 42 that is typically angled at about 45 degrees from the axis of the vascular catheter 40 and is about 1.5 centimeters in length. Vascular catheter 40 may further include a lubricious coating on its inner and/or outer surfaces.

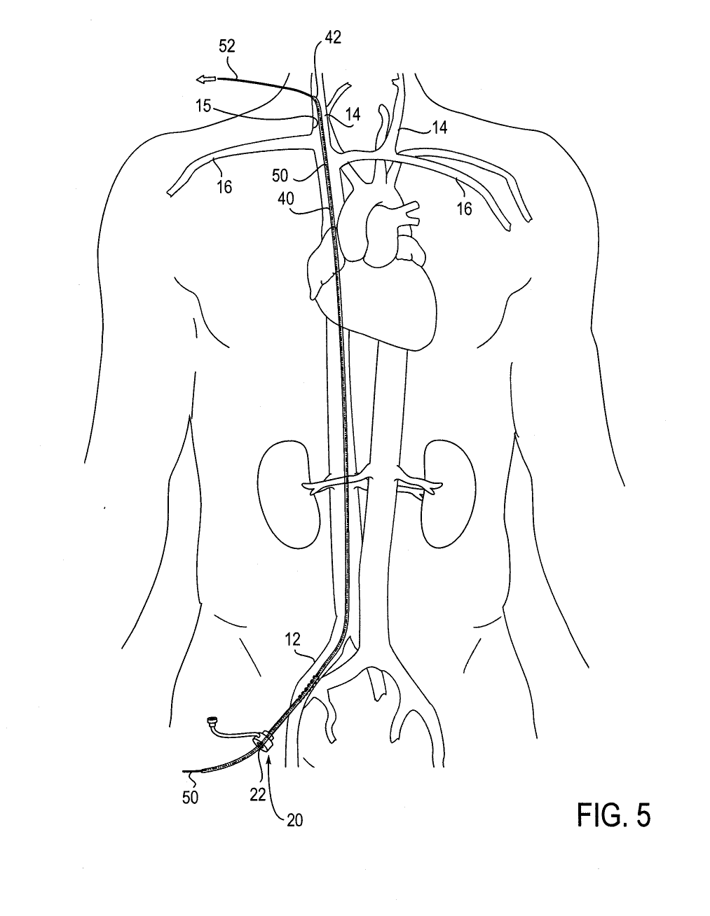

Continuing with embodiments of the method, as shown in FIG. 4, the vascular catheter 40 may then be positioned in the jugular vein 14 with the angled-tip 42 facing the interior wall 15 of the jugular vein 14 at a desired exit site 25. As depicted in FIG. 5, once the vascular catheter 40 is properly positioned, under continuing diagnostic imaging, as in some embodiments, a needle or other similarly configured sharp-tipped device or otherwise penetrating device, such as a needle-tip portion 52 of a stiff intravascular tissue-penetrating guidewire 50, may be passed through the end of the angled-tip 42 of the vascular catheter 40 and further pass through or penetrate the wall 15 of the jugular vein 14. Thereafter, the vascular wall penetrating device such as the needle, or the needle-tip portion 52 and a length of the needle-tipped guidewire 50 are passed through the subcutaneous tissue and exited through the skin of the patient (typically at mid-neck area when the central blood vessel punctured is a jugular vein 14) where they are recovered and pulled above the surface of the skin, as shown in FIG. 5. The vascular catheter 40 may then be removed from the vascular system of the patient, leaving the needle-tipped guidewire 50 in position.

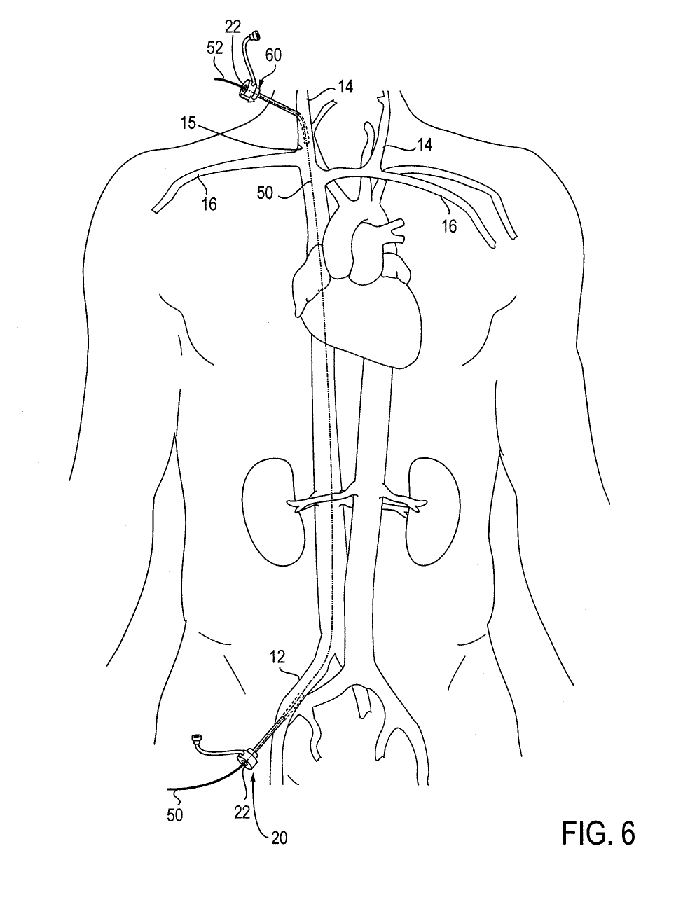

As shown in FIG. 6, a secondary vascular catheter 60 may then be passed over the needle or needle-tip portion 52 and the length of the needle-tipped guidewire 50 such that a distal end of the secondary vascular catheter 60 is placed within the jugular vein 14 while a proximal end remains exposed from the skin of the patient. Thereafter, as shown in FIG. 7, the femoral vascular sheath 20 and the needle-tipped guidewire 50 of the present invention are removed from the patient while the secondary vascular catheter 60 remains in place, now representing a central vein access port. Pressure is then applied over the femoral venapuncture site for several minutes.

In some embodiments, the method of transvascular retrograde access placement includes the use of an angled-tip vascular catheter 41 that has a second channel 46 in addition to a first or a main channel 45. Embodiments of his second channel 46 in the vascular catheter are configured in a diameter of at least about that of the external diameter of a 22-gauge needle (about 0.711 mm). In this method embodiment, the vascular catheter 41 with the second channel 46 is first passed over the general use guidewire 30 and positioned in the jugular vein 14 or other central blood vessel. Next, the general use guidewire 30 is removed from the vascular catheter 41 and a curved-tip stiff, solid obturator 70, having, for example, a relatively fixed curved-tip 72 of about 45 degrees from the axis of the obturator is introduced into the second channel 46 and advanced forward to the distal end of the catheter that is in position at a desired vascular exit and re-entry site 25.

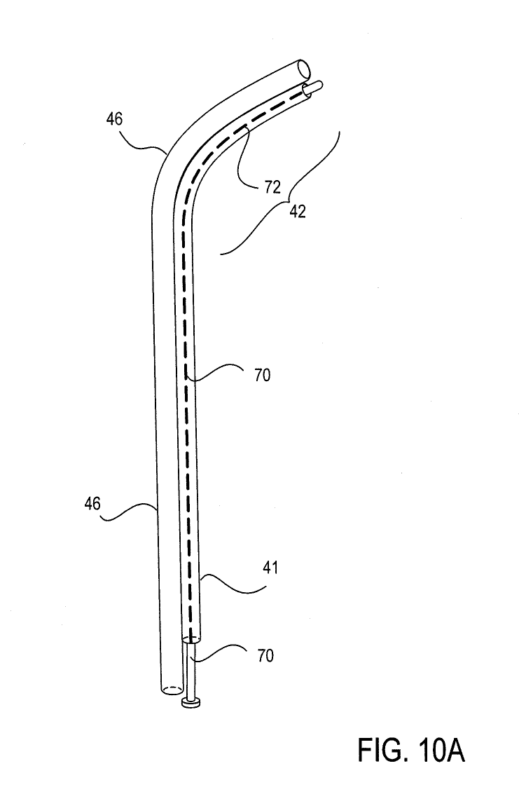

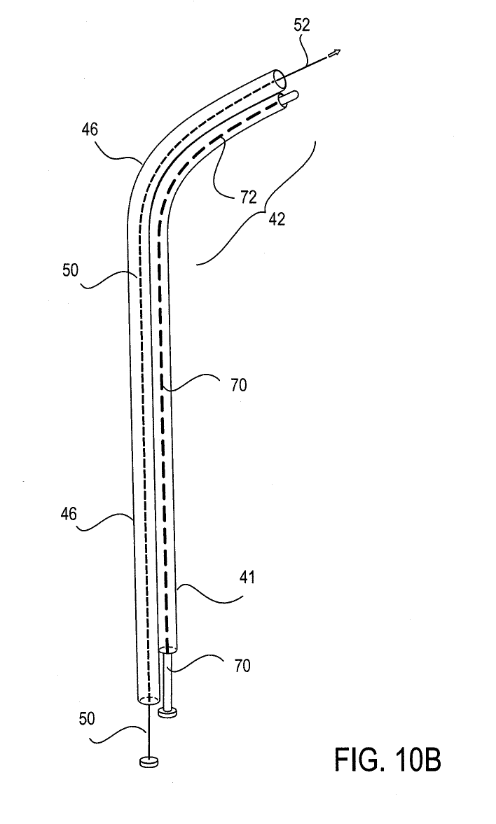

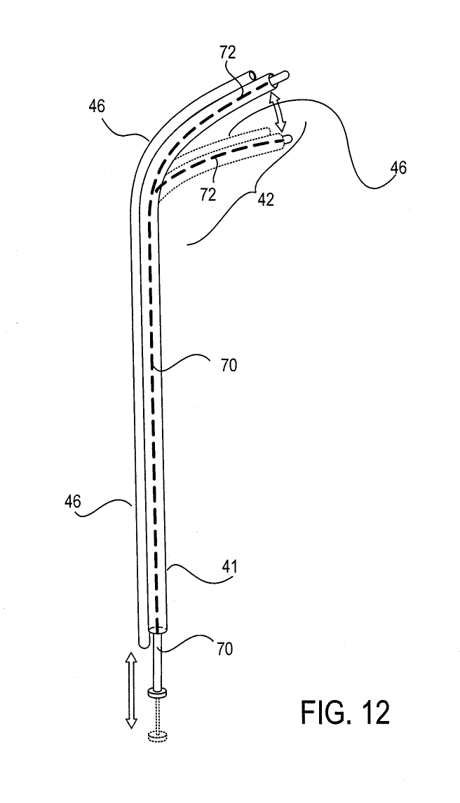

Embodiments of the vascular catheter 41, as mentioned above, may include an angled-tip 42, with a predefined or preferred angle (for example, about 45 degrees from the axis of the catheter). Some embodiments of the method of the invention make use of an angled obturator (FIGS. 10A and 10B, and 12). While using a vascular catheter with an initial or preferred angle may be advantageous, in some embodiments, the tip of the vascular catheter may have a minimally-angled angle portion, which is nevertheless flexible and easily altered by the internal presence of an angled obturator. In embodiments that make particular use of an obturator, the angled tip 42 of the vascular catheter may be formed of materials allowing degrees of flexibility, may bend, thereby further varying the angle thereof from the central axis of the catheter 41 (between, for example about 45 degrees and 90 degrees) by means of and dependent on the length of insertion of the curved-tip 72 of the obturator 70 into the catheter 41 and the tip 42 thereof, as shown in FIG. 12. For example, assuming a 45 degree angled tip 42 and a 45 degree curved tip 72, when the obturator 70 is fully inserted into the vascular catheter 41 such that the curved-tip 72 is passed through from the end of the angled-tip 42, the curved-tip 72 corresponds with the angled-tip 42, thereby maintaining the 45 degree angle of the angled-tip 42. However, when the obturator 70 having a preferred angle of about 90 degrees is partially inserted into or withdrawn from the angled-tip 42, the greater the degree of the angle of the angled-tip 42 becomes, up to, in this example, a 90-degree angle. This embodiment aids in the transvascular retrograde access placement method by enabling the surgeon to penetrate through the wall 15 of jugular vein 14, or other central blood vessel, at a particular angle ranging, in this example, from 45 to 90 degrees.

Once the modified vascular catheter 41 with the curved-tip obturator 70 assembly is properly positioned in the jugular vein 14 at a desired exit site 25, a needle or a stiff intravascular guidewire 50 having a needle-tip portion 52 may be inserted into the second channel 46 of vascular catheter 41. The angled-tip 42 of the catheter 41 is then oriented to face the wall 15 of the jugular vein 14. The needle-tip portion 52 of the needle-tipped guidewire 50 may then pass through the tip of the exterior channel 46 of the modified vascular catheter 41 and puncture the inner wall 15 of the jugular vein 14. Thereafter, the needle-tip portion 52 and a length of the needle-tipped guidewire 50 may be passed up through the subcutaneous tissue, and exited through the skin, and the procedure continues as hereinbefore described.

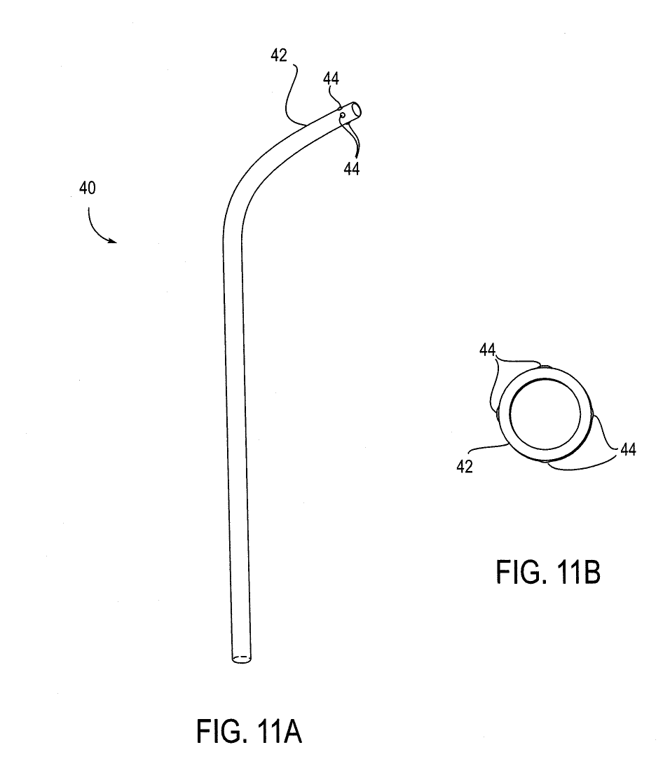

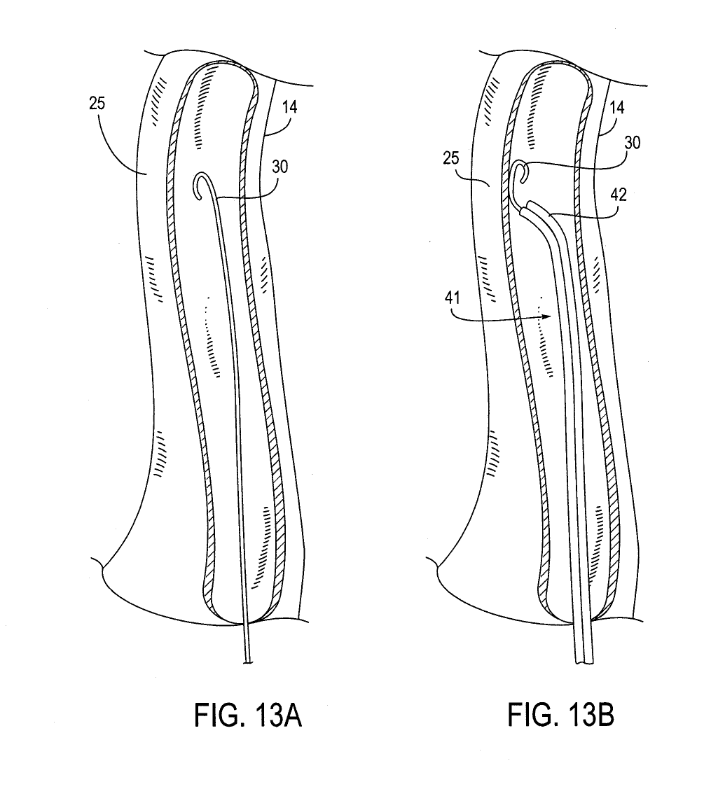

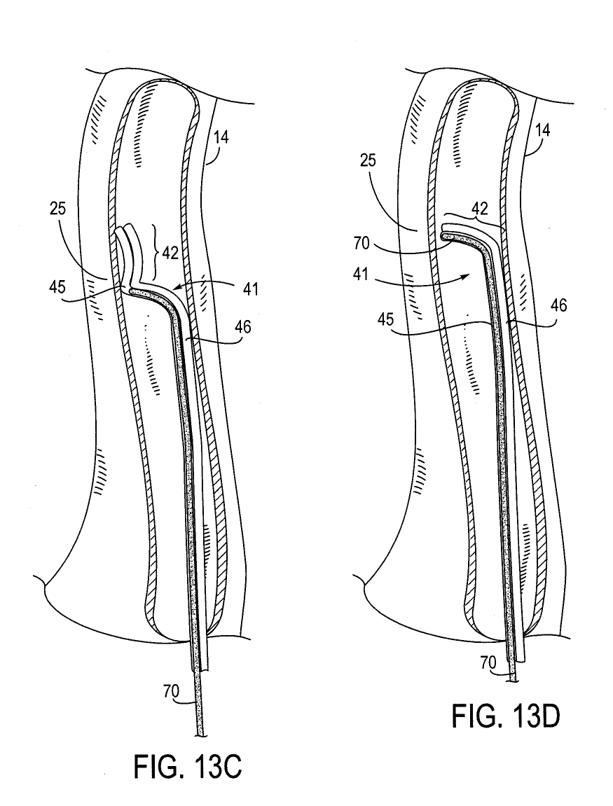

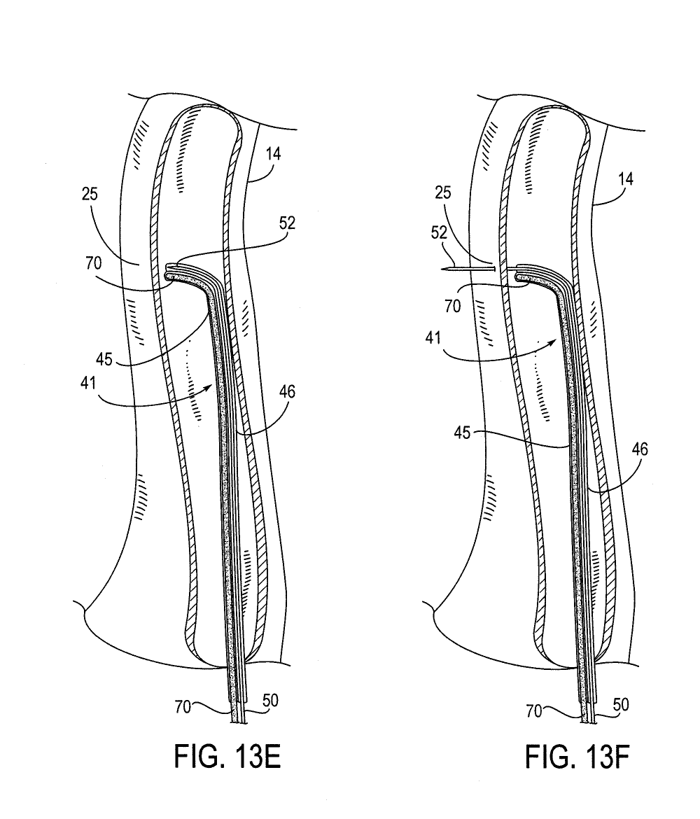

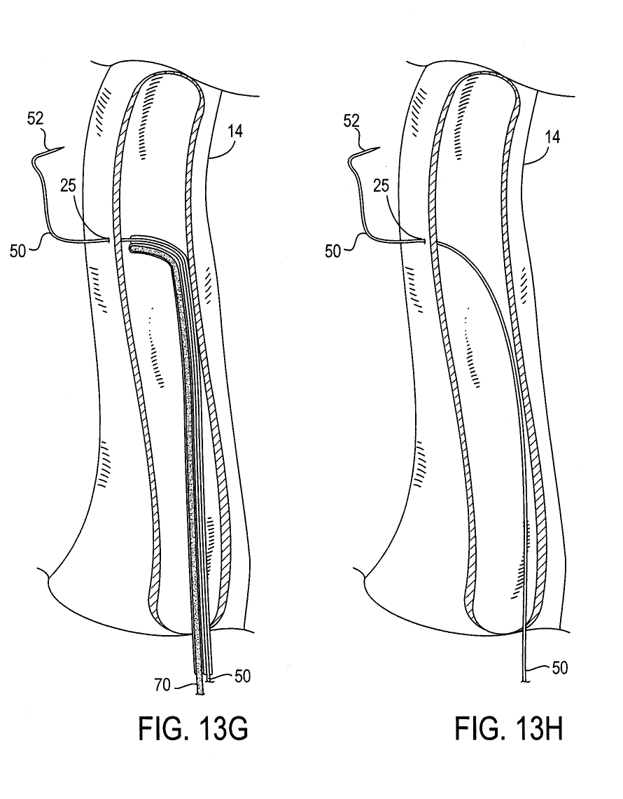

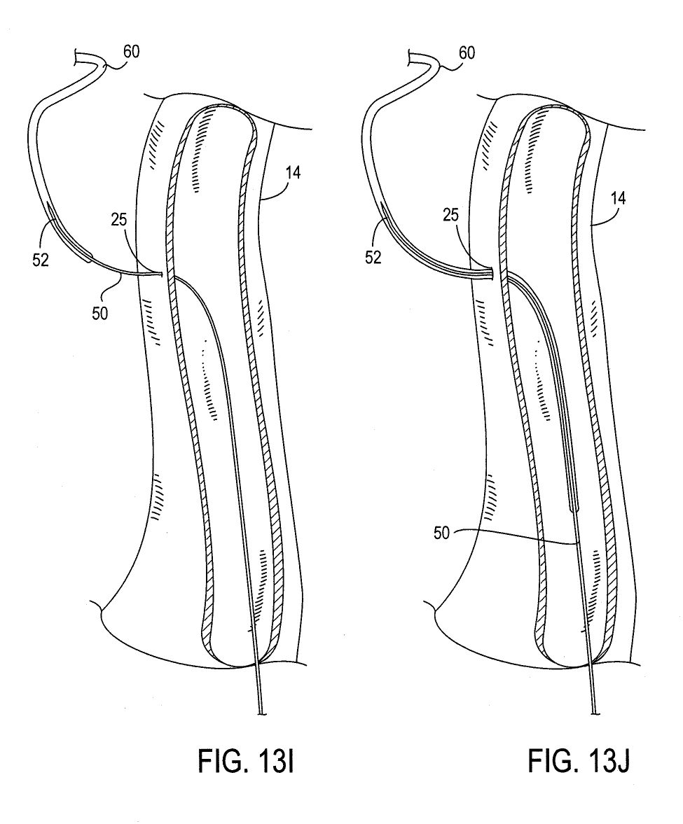

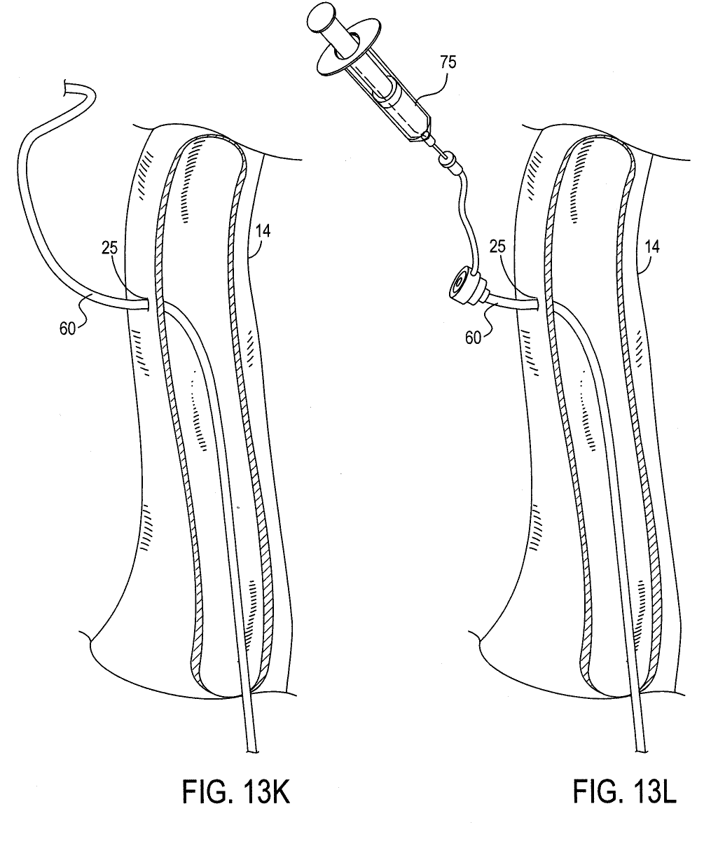

FIGS. 13A-13L provide a view of an embodiment of the method that makes use of a two-channel, angled-tip vascular catheter, with a focus on the activity at the desired site of exit of a tissue penetrating guidewire from a central vein, such as a jugular vein, and the re-entry into the central vein by a vascular access catheter in a downstream vascular direction. These figures show a short section of a jugular vein that includes a desired site for the placement of an access catheter; the figures are oriented with the cephalad end up and caudal end down. The jugular vein is shown with an open window that provides a view into the interior of the vein. FIG. 13A shows the approach of a general use guidewire 30 to the vicinity of a desired vascular exit or pass-through site 25 in a jugular vein 14. FIG. 13B shows the approach of an angled-tip two-channel vascular catheter 41 by way of its primary channel 45 over a general use guidewire 30 to the vicinity of the desired exit site 25 in the jugular vein. The tip of the vascular catheter has an angle of about 45 degrees from the central longitudinal axis of the catheter, and is diagnostically visualizable by the radiopaque marking (see FIGS. 11A and 11B).

FIG. 13C shows the two-channel angled-tip vascular catheter 41 with the general use guidewire 30 having been withdrawn, and a solid wall obturator 70 with a curved tip having been advanced forward in the same primary channel 45 toward the distal end of the vascular catheter 41. FIG. 13D shows a solid obturator 70 having advanced to the distal end of the primary channel 45 of the two-channel vascular catheter 41, the curved tip 42 of the catheter now substantially conforming to the approximate 90 degree angle of the obturator, and perpendicularly facing the wall of the jugular vein at the desired exit site 25.

FIG. 13E shows a stiff guidewire 50 with a penetrating device on the distal end having been advanced to the distal end of the secondary channel 46 of the two-channel vascular catheter 41. FIG. 13F shows the penetrating device 52 mounted on the distal end of the stiff guidewire 50 advancing out of the second channel 46 of the two-channel vascular catheter 41, and penetrating outwardly through the wall of the jugular vein 14 into neck muscle and subcutaneous tissue. FIG. 13G shows the penetrating device 52 penetrating further outward through the skin of the neck and from the body, the stiff intravascular guidewire 50 trailing behind.

FIG. 13H shows the penetrating device 52 and guidewire 50 positioned outside the body, the two-channel vascular catheter 41 now having been withdrawn back through the jugular vein and eventually to exit through its femoral vein site of entry (see FIG. 4). FIG. 13I shows a secondary vascular catheter 60 being passed over the stiff intravascular guidewire 50, now outside the body, in a retrograde direction, from the distal end of the guidewire toward the proximal end. In some embodiments of the method, the penetrating device 52 is removed from the distal end of the guidewire 50 at this point.

FIG. 13J shows the secondary vascular catheter 60 entering the jugular vein 14 in a retrograde direction (in the direction of venous blood flow) over the stiff intravascular guidewire 50. FIG. 13K shows the secondary vascular catheter 60 in place in the jugular vein 14 and positioned to act as a vascular access port, the stiff intravascular guidewire having been withdrawn from the jugular vein, ultimately to exit through its femoral vein site of entry. FIG. 13L shows the secondary vascular catheter 60 in place in the jugular vein 14 as a vascular access port, and as an example of use, a syringe 75 centrally administering drug through the central vein access port.

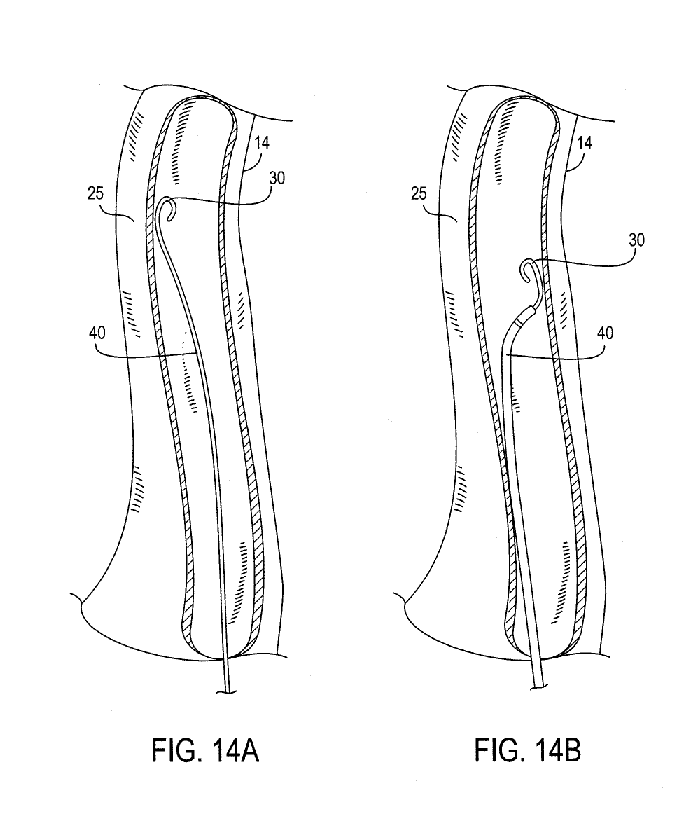

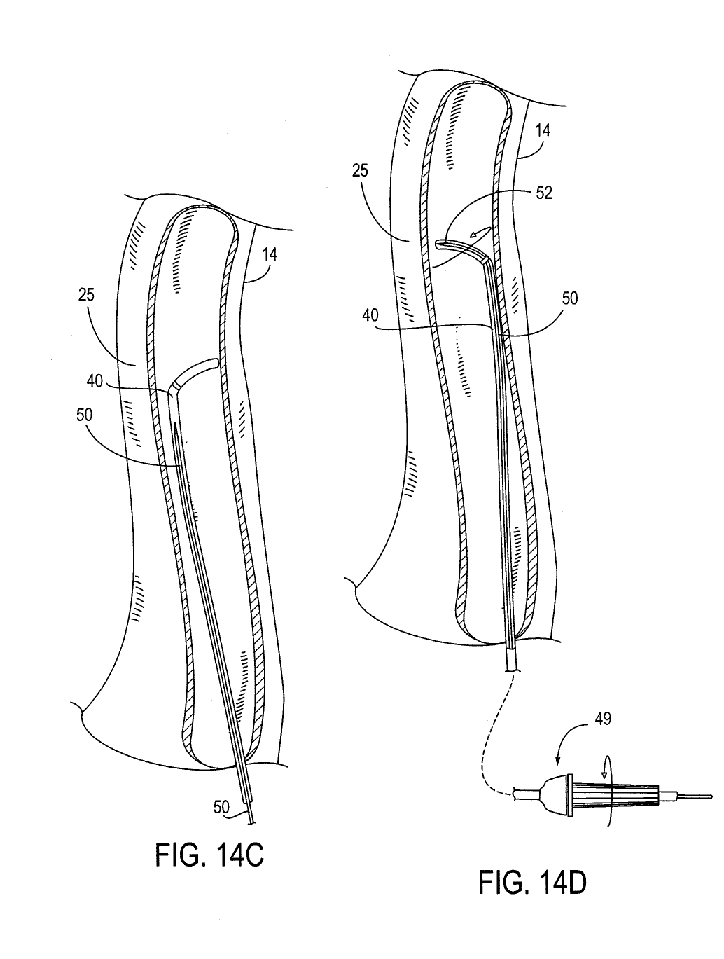

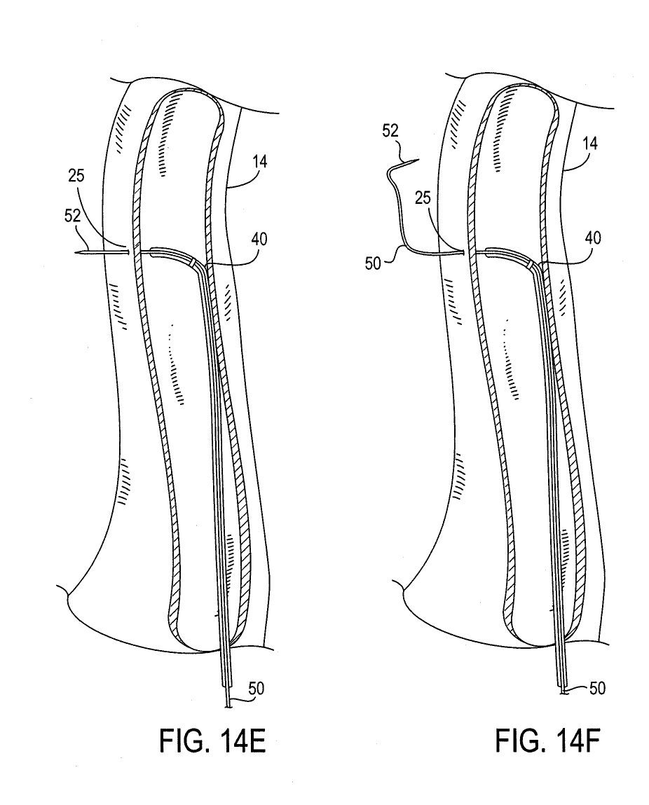

FIGS. 14A-14E show a variation on the method as depicted in FIGS. 13A-13L in which a single channel angled-tip catheter 40 is utilized rather than a two channel catheter. FIGS. 14A-14F focus on the activity that occurs at the site of exit from the jugular vein and re-entry there into. FIG. 14A shows the approach of a general use guidewire 50 to the vicinity of a desired exit site in the jugular vein. FIG. 14B shows the approach of a vascular catheter 40 over the general use guidewire 30 to the vicinity of the desired exit site 25 in the jugular vein 14. The curved or angled tip 42 of the vascular catheter has an angle of about 45 degrees from the central longitudinal axis of the catheter, and may be diagnostically visualizable by the radiopaque marking.

FIG. 14C shows the angled-tip vascular catheter 40 with the general use guidewire 50 having been withdrawn from the catheter, and a stiff guidewire with a penetrating device 52 on its distal end having been advanced to the distal end the curved tip vascular catheter. FIG. 14D shows the angled vascular tip catheter 40 rotating toward a desired exit site on the wall of the jugular vein. The site of vascular exit is on a body exterior-facing aspect of the vein, so the penetrating device that eventually is expressed through the catheter is directed toward the skin, and not internally into the neck. The rotation is being driven by torque applied by a physician to a torquable handle 49 on the catheter at its proximal end.

FIG. 14E shows the penetrating device 52 mounted on the distal end of the stiff guidewire 50 advancing out of the distal opening of the vascular catheter and directly into the wall at the exit site 25, penetrating outwardly through the wall of the jugular vein into neck muscle and subcutaneous tissue. FIG. 14F shows the penetrating device penetrating further outward through the skin of the neck and from the body, the stiff intravascular guidewire trailing behind. Hereinafter, the method proceeds as depicted in FIGS. 13H-13L, wherein, briefly, as the penetrating device and attached guidewire are outside the body, a secondary vascular catheter is passed in a retrograde direction over the guidewire, through the exit site, and into the jugular vein. In an optional step, the penetrating device may be removed from the guidewire prior to the secondary vascular catheter being passed there over. After insertion of the vascular catheter, the guidewire is removed from the jugular vein, typically through its site of entry in the femoral vein, and the secondary vascular catheter remains in place as a central vein access port.

In another variation of the method, an embodiment of a penetrating device, such as an elongate curved needle may be inserted through an angle-tipped vascular catheter to form an opening or pass-through which traverses from a central blood vessel to the skin, and the needle is then withdrawn back into the catheter and removed proximally therefrom. The catheter remains in place, and a guidewire is then advanced distally through the catheter, through the pass-through formed by the needle, and exiting from the body through the skin overlaying the vein. Thereafter, the initial vascular catheter is withdrawn from the site, and a secondary vascular catheter is threaded over distal end the guidewire (free, outside the body) and into the central vein in a retrograde manner as seen in FIGS. 13H-13L. The method embodiment just described may be understood as having two particular steps (after appropriately positioning the initial vascular catheter) to establish a guidewire in place over which a vascular access device (a secondary vascular catheter): (1) a needle forms a vascular pass-through, and (2) a guidewire is threaded through the pass-through. This method may have advantages in particular medical applications; however, also provided herein is a method in which these two steps are combined into a single step.

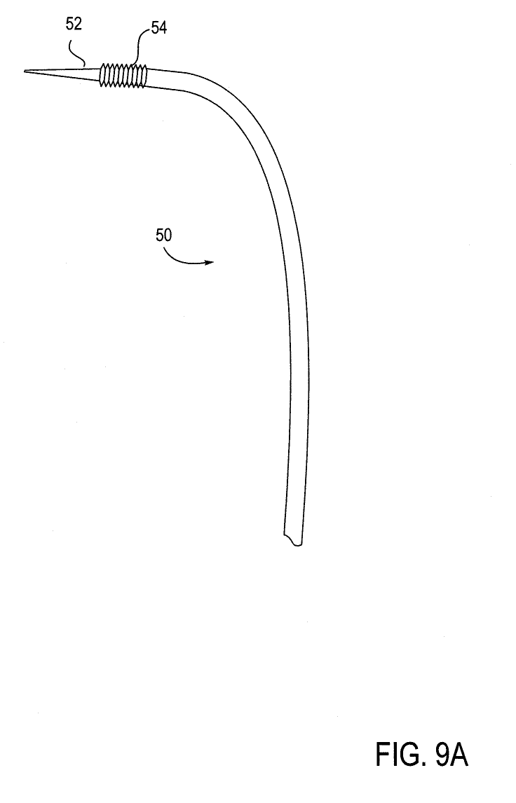

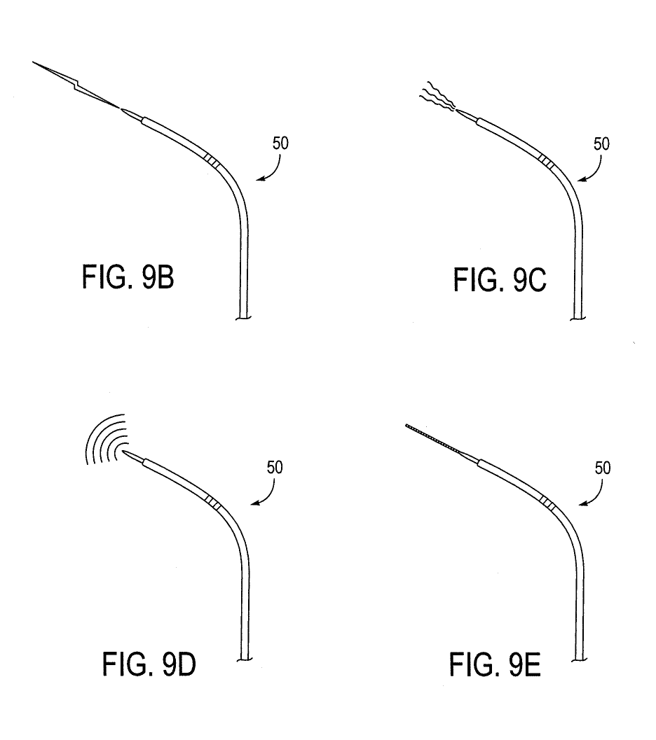

The use of a needle-tipped guidewire, as described here in, and as shown in method steps depicted in FIGS. 4-6, 8, 13E-13J, and as depicted as device embodiments in FIGS. 9A-9E, may be understood as a method that combines the just-recited two steps into a single step that provides a guidewire in place. In this “single-step” embodiment, a penetrating device mounted on the distal end of a stiff guidewire passes through the vascular wall and skin with a guidewire trailing behind. The advantages of this embodiment include the reduction in steps (reducing two steps to a single step) and a reducing the number of operating implements from two (needle and guidewire) to one (needle-tipped guidewire).

Other embodiments of the methods of transvascular retrograde access placement procedure may be performed in a central blood vessel other than the jugular vein 14, wherein the needle, the needle-tipped guidewire 50, or any other similar device, may exit the patient through another area of the patient. For example, the transvascular retrograde access placement procedure may be performed in a subclavian vein 16, wherein a needle, a needle-tipped guidewire 50, or other similar device exits the patient through skin of the upper trunk just below a clavicle. In still other embodiments of the method, a blood vessel other than a central vein, such as a peripheral vein or an artery, may be accessed by re-entry into an opening created by outward penetration from within the vessel. The method may be particularly advantageous when external access to the vessel is complicated by normal anatomy (such being the case with the subclavian artery, for example), or by a complicating injury or medical condition.

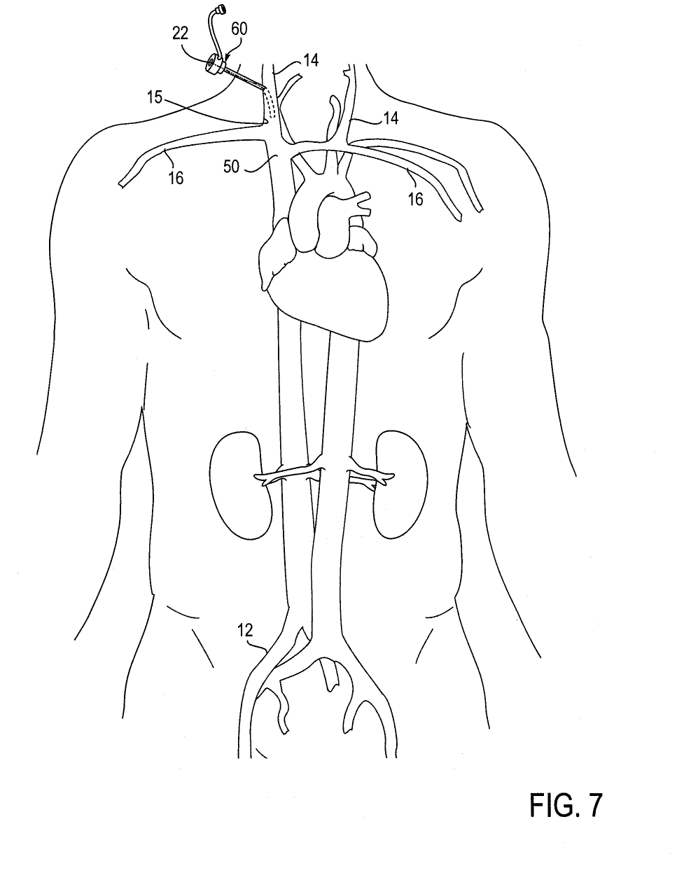

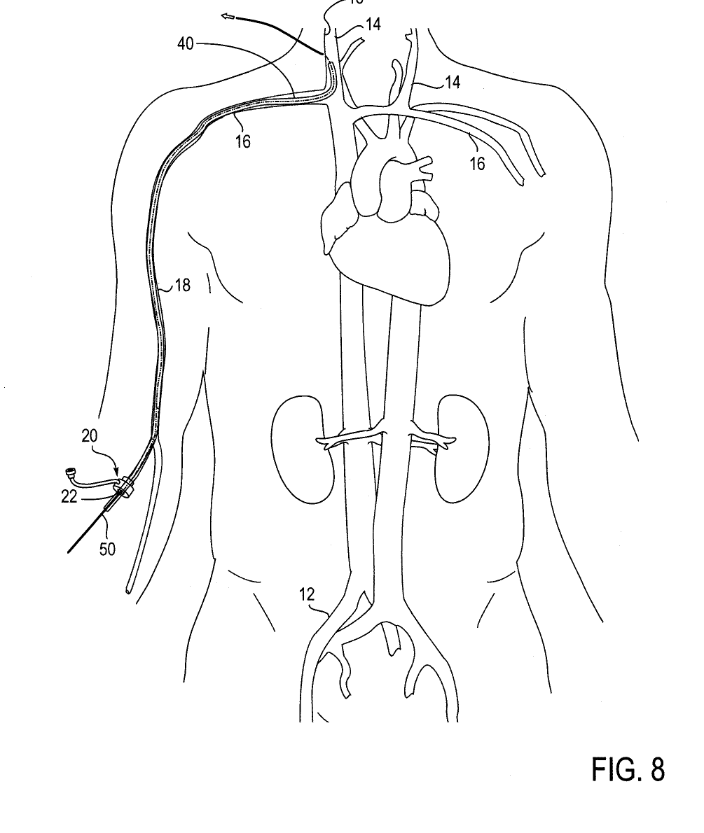

Another embodiment of the method of transvascular retrograde access placement varies from the previously described methods in that rather approaching the central vein by way of femoral vein cannulation, an antecubital blood vein or artery (located in the antecubital fossa of an arm) is cannulated. As shown in FIG. 8, this method embodiment more specifically includes, through the application of the Modified Seldinger Technique and the insertion of a vascular sheath 20, inserting a general use guidewire 30 into an antecubital vein 18 and then passing a vascular catheter 40, per embodiments of the invention, over the guidewire. The vascular catheter 40 is then passed up the vascular system to a jugular vein 14, or a subclavian 16 vein, or any other central vein. The procedure thus generally follows the steps depicted in FIGS. 2-7, except for the entry through the antecubital vein rather than the femoral vein. After the moment depicted in FIG. 8, the remainder of this method embodiment typically proceeds in accordance with the previously described embodiments of the present invention, as shown, for example, in FIGS. 6 and 7.

As has been noted in the description focused on embodiments of the method of transvascular retrograde access placement, the present invention also provides devices and device features, some of which will now be described in greater detail. One such device, as shown in FIG. 9A, is a stiff intravascular guidewire 50 that includes a needle-tip portion 52 at one end thereof, typically ranging in length from at least about 1 cm to about 2.5 cm; the length is advantageously commensurate with the depth of tissue that the needle needs to pass through from the wall of the vein to the surface of the skin, where it can be grasped by an instrument and pulled through. The 1 cm to 2.5 cm range approximates the range of tissue depth above the jugular vein. In applications of the invention to other vascular sites, the length of the needle may be modified appropriately. The needle-tipped guidewire 50 may further include a lubricious coating on its exterior surface. The needle-tipped guidewire 50 may also or alternatively be configured such that the needle-tip portion 52 may be removable from the remainder of the guidewire 50, as for example, by means of a screw/thread mechanism 54 similar to that utilized with the Amplatzer vascular plug occluder detachment system.

As previously noted, the scope of the tissue-penetrating guidewire 50, as described herein, includes any conventional device that can penetrate tissue with an appropriate level of precision and control, and may be sized and adapted to be supportable at the distal end of a stiff guidewire, as described herein. The mechanism of attachment or coaption to a guidewire may be by way of any conventional joining method, including fixed-connection methods, such as welding, soldering, or gluing, or removable-connection methods, such as threading, hinging, or bayonet mounting. Thus, FIGS. 9A-9D show various embodiments of a stiff intravascular guidewire with a penetrating device mounted at the distal end. FIG. 9A shows a needle-tipped guidewire. FIG. 9B shows a guidewire with a distally-mounted radiofrequency knife. FIG. 9C shows a guidewire with a distally-mounted electrosurgical device. FIG. 9D shows a guidewire with a distally-mounted high frequency ultrasound emitter. FIG. 9E shows a guidewire with a distally-mounted laser device. The devices are typically powered by a generator, energy being conveyed in some embodiments (e.g., radiofrequency knife and electrosurgical device) by the guidewire itself, or particular strands braided into or along side the guidewire. In other embodiments (e.g., ultrasound emitter, laser), dedicated energy-deliver connections may be aligned along side or attached to the guidewire.

Embodiments of this needle-tipped guidewire 50 typically have a length of at least about 260 centimeters, but it in other embodiments of the present invention, the guidewire may have a length that varies between of about 150 cm to about 300 cm. Embodiments of this needle-tipped guidewire 50 may be configured in a variety of diameters, such as, for example, about 0.035 inches, 0.018 inches, or 0.014 inches, as may be appropriate for use in a variety of catheterization procedures involving blood vessels other than central blood vessels.

Some embodiments the vascular catheter 40 include two channels, a first channel 45 and a second channel 46 as shown in FIGS. 10A, 10B, 12, and 13A-13L. As can be seen with reference to these figures, the relative positions of the first and second channels at the angled tip portion are such that the first channel is inferior and the secondary channel is superior. The second channel 46 generally is configured with an inner diameter of at least about that of a 22-gauge needle. As shown in FIG. 10B, the second channel 46 serves as a guide for the insertion of a needle, or other similarly configured device, or a needle-tipped guidewire 50 of the present invention, that may be used to penetrate a central blood vessel outwardly from its interior. Embodiments of this modified vascular catheter 40 and the second channel 46 may be provided in different sizes configurations as may be appropriate in their application to various other blood vessels.

Another feature of devices that facilitates performance of described methods relates to the modification of an angled-tip vascular catheter 40 to include one or more diagnostically-opaque markers 44 that may be positioned within or about the angled-tip 42 of the vascular catheter 40, as shown in FIGS. 11A and 11B. In a particular embodiment, a vascular catheter 40 is configured with four gold markers 44 positioned about the exterior of the angled-tip 42. The diagnostically-opaque markers 44 may be configured to indicate, through advanced diagnostic imaging technologies, the orientation of the angled-tip 42 of the vascular catheter 40 in relation to a wall 15 of a central blood vessel. In a related embodiment, the angled-tip 42 of the vascular catheter 40 may be provided with diagnostically-opaque markers 44 dispersed within the material forming the angled-tip 42. These various types of opaque markers can facilitate a procedure by allowing visualization of the orientation of the angled-tip 42 of the vascular catheter 40, through which a penetrating device, such as a needle-tipped guidewire 50, may be passed toward the wall of the vessel, and to ensure the proper alignment of the angled-tip 42 with the wall 15 of the central blood vessel prior to penetrating the wall.

Unless defined otherwise, all technical terms used herein have the same meanings as commonly understood by one of ordinary skill in the art of vascular catherization. Specific methods, devices, and materials are described in this application, but any methods and materials similar or equivalent to those described herein can be used in the practice of the present invention. While embodiments of the invention have been described in some detail and by way of exemplary illustrations, such illustration is for purposes of clarity of understanding only, and is not intended to be limiting. Various terms have been used in the description to convey an understanding of the invention; it will be understood that the meaning of these various terms extends to common linguistic or grammatical variations or forms thereof. Moreover, any one or more features of any embodiment of the invention can be combined with any one or more a features of any other embodiment of the invention, without departing from the scope of the invention. Still further, it should be understood that the invention is not limited to the embodiments that have been set forth for purposes of exemplification, but is to be defined only by a fair reading of claims that are appended to the patent application, including the full range of equivalency to which each element thereof is entitled.

1.-36. (canceled)

37. A device suitable for use in performing transvascular retrograde access placement comprising:
a vascular catheter comprising a channel having an inner diameter of at least about that of an external diameter of a 22-gauge needle;
a stiff intravascular guidewire disposed in the channel, the guidewire having a diameter greater than or equal to 0.014 inches and less than or equal to 0.035 inches; and
a vascular wall penetrating device supported on a distal end of the guidewire;
the vascular catheter, intravascular guidewire and vascular wall penetrating device being further adapted to cooperate to pass the penetrating device through a vascular wall, subcutaneous tissue and skin at an exit point of a patient.

38. The device of claim 37, wherein the vascular wall penetrating device is removably coupled to the guidewire.

39. The device of claim 38, wherein the vascular wall penetrating device is removably coupled to the guidewire with a threaded mechanism.

40. The device of claim 37, wherein the penetrating device is a needle.

41. The device of claim 40, wherein the needle has a length in the range of at least about 1 cm to about 2.5 cm.

42. The device of claim 37, wherein the penetrating device is a radiofrequency knife.

43. The device of claim 37, wherein the penetrating device is a laser.

44. The device of claim 37, wherein the penetrating device is a high frequency ultrasound device.

45. The device of claim 37, wherein the penetrating device is an electrosurgical device.

46. The device of claim 37, wherein the guidewire has a length that varies between about 150 cm and about 300 cm.

47. The device of claim 37, wherein the guidewire has a length that varies between about 225 cm and about 275 cm.

48. The device of claim 37, wherein the guidewire further comprises a lubricious coating.

49. A device suitable for use in performing transvascular retrograde access placement comprising a vascular catheter comprising an angled tip and two channels.

50. The device of claim 49, wherein at least one of the two channels has an inner diameter of at least about that of an external diameter of a 22-gauge needle.

51. The device of claim 49, wherein the angled-tip comprises an angled portion with an angle of about 30 to about 60 degrees with respect to the longitudinal axis of the catheter.

52. The device of claim 51, wherein the angled portion is sufficiently elastic to become substantially linear when the catheter is within the confines of a blood vessel and to elastically assume a more acute angle when a curved obturator with an angled portion is advanced into the angled portion of the vascular catheter.

53. The device of claim 49, wherein the vascular catheter further comprises one or more diagnostically-opaque markers.

54. The device of claim 53, wherein the diagnostically-opaque markers are positioned within or about the angled-tip of the angled-tip vascular catheter.
